(19) United States
(12) Patent Application Publication
(10) Pub. No.: US 2006/0165687 A1
(43) Pub. Date: Jul. 27, 2006

(54) Vaccine adjuvant

(75) Inventors: Barton F. Haynes, Durham, NC (US); Gregory D. Sempowski, Durham, NC (US); James W. Peacock, Durham, NC (US)

Correspondence Address: NIXON & VANDERHYE, PC, 901 NORTH GLEBE ROAD, 11TH FLOOR, ARLINGTON, VA, 22203, US

(73) Assignee: DUKE UNIVERSITY, Durham, NC (US)

(21) Appl. No.: 11/302,505

(22) Filed: Dec. 14, 2005

Related U.S. Application Data
(63) Continuation of application No. PCT/US05/37384, filed on Oct. 19, 2005.
(60) Provisional application No. 60/619,686, filed on Oct. 19, 2004.

(51) Int. Cl. A61K 39/395

(52) U.S. Cl. 424/143.1

(57) ABSTRACT

The present invention relates, in general, to a method of enhancing an immune response in a mammal and, in particular, to a method of enhancing an immune response to a vaccine comprising suppressing the number and/or function of regulatory T cells. The invention further relates to compounds and compositions suitable for use in such a method.

This application is a continuation-in-part of PCT/US05/37384, filed Oct. 19, 2005, which claims priority from Provisional Application. No. 60/619,686, filed Oct. 19, 2004, the contents of which applications are incorporated herein by reference.

### TECHNICAL FIELD

The present invention relates, in general, to a method of enhancing an immune response in a mammal and, in particular, to a method of enhancing an immune response to a vaccine comprising suppressing the number and/or function of regulatory T cells of the mammal. The invention further relates to compounds and compositions suitable for use in such a method.

### BACKGROUND

T regulatory cells have been identified that suppress B and T cells responses to parasitic infections and viral (e.g., HIV) infections (Messer et al, J. Virol. 78:11641-11647 (2004); Suvas et al, J. Exp. Med. 198:889-901 (2003); Haynes et al, J. Immunol. 123:2095-2101 (1979); Stephens et al, J. Immunol. 173:5008-5020 (2004); Kursar et al, J. Exp. Med. 196:1585-1592 (2002)). These cells constitutively express high levels of FOXP3 (Shevach, Arth. Rheum. 50:2721-2724 (2004)). These cells have been found to down-regulate host responses to anti-cancer immune responses (Shimizu et al, J. Immunol. 163:5211-5218 (1999)). Depletion of T regulatory cells has been suggested as a means for enhancing host anti-tumor responses and for enhancing the effect of anti-tumor immunotherapies (Steitz et al, Cancer Res: 61:8643-8646 (2001); Woo et al, J. Immunol. 168:4272-4276 (2002); Sutmuller et al, J. Exp. Med. 194:823-832 (2001); Onizuka et al, Cancer Res. 59:3128-3133 (2001); Ahlers et al, PNAS 99:13020-13025 (2002)). (See also Brändlein et al, Cancer Res. 63:7995-8005 (2003).)

Removal of T regulatory cells has also been suggested as an approach to improve immunogenicity of “weak” vaccines (Shevach, J. Exp. Med. 193:F41-F45 (2001)). However, with many vaccines, the immune response may be strong but antibodies of the appropriate type and specificity are not induced (e.g., antibody responses to HIV envelope are often against non-neutralizing, rather than neutralizing, determinants on gp160). In addition, many live virus vaccines are in and of themselves immunosuppressive. This induction of suppression of the host immune response results in dampened responses to the vaccine and lowered protection induced by the vaccine—a prime example is the tuberculosis (TB) vaccine, BCG.

The present invention results, at least in part, from the realization that a reason that broadly reactive antibodies of appropriate type and specificity may not be made in response to HIV envelope immunization is due to the similarity that exists between such antibodies and “natural” antibodies (antibodies responsible for innate immunity) that are present in fetal life and that are either constitutively present or are produced in response to environmental antigens (Marchalonis et al, FASEB J. 16:842-848 (2002); Lake et al, Proc. Natl. Acad. Sci. USA 91:10849-10853 (1994)). When a fetus develops into a postnatal infant and then into an adult, these natural antibodies are brought under immunoregulatory control, only to be released in the context of autoimmune disease, or transiently in response to infectious agents that can polyclonally induce B cell activation, such as EB virus infection. Anti-HIV gp160 antibodies constitute one class of such natural antibodies. Braun and co-workers have shown that natural antibodies with VH3 genes are natural ligands for gp120 (Berberian et al, Science: 261:1588-1591 (1993)), demonstrating that genetic lack of these antibodies is a risk factor for HIV 1 tranmission (Townsley-Fuchs et al, J. Clin. Invest. 98:1794-1801 (1996)). Zouali (Appl. Biochem. Biotechnol. 61:149-155 (1996)) has found that HIV infection drives the expansion of VH3 antibodies as a superantigen and has suggested that HIV triggering of these B cells could promote autoimmunity in HIV infection. (See also Metlas et al, Immunol. Letters 47:25-28 (1995); Prljic et al, Vaccine 17:1462-1467 (1999).)

Rare human monoclonal antibodies derived from HIV infected patients have been made from patient B cells that bind to neutralizing determinants on gp160 of HIV Env, and are broadly neutralizing (reviewed in Burton et al, Nat. Immunol. 5(3):233-236 (2004)). However, existing immunogens do not induce these types of antibodies—while immunogens may have these epitopes on their surface, only minimal responses have been reported. The reason for the lack of induction of these types of responses is likely not in the immunogen but in the host. Aandahl et al (J. Virol. 78:2454-2459 (2004)) have shown that T regulatory cells are induced during HIV infection and suppress T cell responses. These authors propose that, in chronic viral (e.g., HIV) infections, manipulation of T regulatory cells could help restore antigen specific immune responsiveness. In contrast to the suggestions of Aandahl et al (J. Virol. 78:2454-2459 (2004)), the present invention results from appreciation of the fact that, in an HIV uninfected subject, B cell clones that give rise to broadly reactive neutralizing antibodies are present early on in life and are in the family of “natural” antibodies, or are similar to them, and thus are under normal T regulatory cell control. This appreciation results in the present approach of achieving induction of broadly reactive neutralizing antibodies with the desired specificities, which approach comprises administering HIV envelope immunogens at the time of transient abrogation or blocking of T regulatory function to “release” the normal immune system to respond to those regions on the HIV envelope to which broadly reactive neutralizing antibodies can be made.

T regulatory cells are also likely involved the myriad of ways that mycobacteria and other intracellular organisms suppress immunity and prevent adequate immune responses to them (Monack et al, Nat. Rev. Microbiol. 2:747-765 (2004)). To either control active infection or in the setting of BCG vaccination, it is likely that T regulatory cells are induced. It is not believed that induction of T regulatory cells per se in TB has been reported, however, it has been reported that a key cytokine produced by T regulatory cells, IL-10, is produced in multiple drug resistant TB (MTB) (Lee et al, Clin. Exp. Immunol. 128:516-524 (2002)).

In accordance with the present invention, patients with MTB can be treated with transient episodes of abrogation of T regulatory cells to enhance immune responses to the pathogen and to assist the patient in clearing the MTB.

### SUMMARY OF THE INVENTION

The present invention relates to a method of enhancing an immune response in a mammal. More specifically, The invention relates to a method of enhancing an immune response to a vaccine comprising suppressing the number and/or function of regulatory T cells. In addition, the invention relates to compounds and compositions suitable for use in the present method.

Objects and advantages of the present invention will be clear from the description that follows.

### BRIEF DESCRIPTION OF THE DRAWINGS

FIG. 1. Kinetics of T-reg regeneration following high range PC61 and Y13 treatment.

FIG. 2. Kinetics of T-reg regeneration following low range PC61 and Y13 treatment.

FIG. 3. Serum antibody titers for T-Reg depleted and gp140 immunized BALB/c mice.

FIG. 4. Gp140 specific IFN-γ spot forming cells from animals treated with PC61 or Y13.

FIG. 5. T regulatory cell modulation of HIV-1 experimental vaccine immunogen induced IFN-γ spleen spot forming cells in BALB/c mice.

FIGS. 6A and 6B. Timing of appearance of new CD4⁺/CD25⁺ T cells following depletion with PC61 Mab. (FIG. 6A) Thymus. (FIG. 6B) Peripheral blood.

FIG. 7. Recovery of CD4⁺/CD25⁺ Foxp3+ cells in spleen.

FIGS. 8A and 8B. Impact of T regulatory cell removal (PC61 depletion v. Mab Y13 control) on BALB/c immune response to M. smegmatis. FIG. 8A. Systemic (spleen). FIG. 8B. Mucosal (lung).

FIG. 9. Frequency of T regulatory phenotype T cells in non-human primate depletion model.

FIG. 10. Recovery of T-regulatory phenotype cells in whole blood is dose dependent.

FIG. 11. Immunization protocol.

FIG. 12. T-regulatory depletion does not significantly alter the tetramer specific CD8+ profile of splenocytes.

FIG. 13. T-regulatory depletion does not significantly alter the tetramer specific CD8+ profile of lung lymphocytes.

FIG. 14. Depletion of T-regulatory cells enhances peptide-specific IFN-γ SFC responses in the spleen.

FIG. 15. Depletion of T-regulatory cells enhances peptide-specific IFN-γ SFC responses in the lungs.

FIGS. 16A and 16B. T-regulatory recovery in the spleen. FIG. 16A. Percent of T-regulatory cells. FIG. 16B. Absolute number of T-regulatory cells.

FIGS. 17A and 17B. T-regulatory recovery in the thymus. FIG. 17A. Percent of T-regulatory cells. FIG. 17B. Absolute number of T-regulatory cells.

FIG. 18. Ribi.

FIGS. 19A-19D. T-regulatory recovery after treatment with Ribi. FIG. 19A. Percent positive thymocytes in the thymus. FIG. 19B. Absolute number in the thymus. FIG. 19C. Percent positive splenocytes in the spleen. FIG. 19D. Absolute number in the spleen.

FIG. 20. TLR-ligands studied and approach used.

FIGS. 21A-21C. FIG. 21A. Recovery of percent Foxp3+, CD25+, CD4 single positive thymocytes. FIG. 21B. Day 10. FIG. 21C. Day 17.

FIGS. 22A-22C. FIG. 22A. Recovery in absolute number of Foxp3+, CD25+, CD4 single positive thymocytes. FIG. 22B. Day 10. FIG. 22C. Day 17.

FIGS. 23A-23C. FIG. 23A. Recovery of percent Foxp3+, CD25+, CD4+ splenocytes. FIG. 23B. Day 17. FIG. 23C. Day 24.

FIGS. 24A-24C. FIG. 24A. Recovery of absolute number Foxp3+, CD25+, CD4+ splenocytes. FIG. 24B. Day 17. FIG. 24C. Day 24.

FIG. 25. Number of thymocytes isolated on day 10 post treatment with Y13 (0.25 mg) and TRL agonists.

### DETAILED DESCRIPTION OF THE INVENTION

Safely amplifying immune responses to vaccines is an important goal of development of emerging infection vaccines. The present invention provides a method to achieve such amplification. This method comprises suppressing CD4+/CD25+ T-regulatory (T-reg) cell number and/or function at the time of vaccination. The suppression effected in accordance with the invention is transient in nature, not chronic, followed by recovery to normal (e.g., pre-suppression) levels of CD4+/CD25+ T-regulatory cell number/function (e.g., within about 3 days to 6 weeks). Advantageously, the transient suppression of the present method is acheived without interfering with immuno-surveillance afforded by other T regulatory cell-types.

The present invention results from the appreciation that immunoglobulins that are made in response to broadly reactive neutralizing epitopes (e.g., of HIV envelope) may not be routinely made because they are a member of a family of primoridial genes that are stimulated by other antigens (environmental antigens, host antigens, DNAs, etc) and are potentially autoreactive. These immunoglobulins are seen by the body as autoantibodies and systems exist to keep such potentially damaging antibodies under control. Thus, the invention provides for the transient abrogation of T regulatory cells in immunizations. In preferred embodiments, the present approach is used in the context of HIV vaccines and in the context of TB vaccines for both T and B cell response to TB and for recombinant BCG and attenuated TB in order to afford better immunogens.

The T-regulatory cells suppressed in accordance with the present method are CD4+/CD25+ regulatory T cells. These cells constitutively express high levels of Foxp3 (Shevach, Arth. Rhem. 50:2721-2724 (2004)). Suppression can be effected by depleting the number of such cells or inhibiting the function of these cells as immune suppressors.

Depletion of the number of T-regulatory cells can be effected using any of a variety pharmaceutically acceptable agents, including small molecules and antibodies (e.g., monoclonal antibodies, preferably, humanized monoclonal antibodies). Antibodies that bind specifically to the alpha subunit (p55 alpha, CD25, or Tac subunit) of the human high-affinity interleukin-2 receptor that is expressed on the surface of activated lymphocytes are particularly preferred, ZENAPAX (daclizumab) being a specific example. Alternatively, diphtheria toxin conjugated to IL-2, such as ONTAK, can be used (e.g., in humans) to effect transient depletion of T regulatory cells.

Suppression of the immune suppressor function of CD4+/CD25+ cells can be effected, for example, by inhibiting Foxp3 expression (the genomic sequence for FOXP3 is found at Genbank Accession No. AF235087 (see also U.S. application Ser. No. 09/372,668 which discloses the cDNA sequence)). Expression altering agents include small molecules, peptides, polynucleotides (e.g. siRNA's that target Foxp3), cytokines, and antibodies (or fragments thereof) (see U.S. application 20030170648). Further, blocking cell surface molecules (e.g., CTLA4 and GITR) required for function of T regulatory cells can be used. Suitable blocking agents include siRNAs that target these cell surface molecules, antibodies specific for such molecules. Blocking agents can also be small molecules or proteins, plasmids expressed in vaccine vectors or plasmids administered as DNAs.

The agent(s) used to effect suppression of the CD4+/CD25+ cells can be co-administered with the immunogen (vaccine) or shortly before (e.g., about 1-14 days, preferably 1-7 days, more preferably, 1-4 days) administration of the immunogen. Administration shortly after immunization may be effective under certain circumstances. Optimum regimens can be determined by one skilled in the art and can vary with the agent, the immunogen, the patient and/or the specific effect sought.

In one preferred embodiment, the immunogen administered can be one or more HIV envelope peptides/proteins that induce broadly reactive neutralizing antibodies (similar to broadly reactive neutralizing antibodies 2F5, 4E10, 1b12, and 2G12 (Wolbank et al, J. Virol. 77:4095-4103 (2003); Kunert et al, AIDS Res. Hum. Retro. 20(7):755-762 (2004)), or nucleic acids encoding same. Centralized (e.g., consensus, ancestral or center of the tree) sequences can be used as the immunogen (e.g., including sequences disclosed in PCT/US04/30397), as can mosaic proteins (e.g., including proteins disclosed in U.S. Provisional Appln. 60/710,154).

In another preferred embodiment, the immunogen can be a mycobacteria vaccine, such attenuated TB, BCG, or BCG expressing exogenous genes (e.g. HIV genes or other genes that enhance BCG immunogenicity (such as listerolysin)). In accordance with the present method, patients with MTB can be treated with doses of agents that transiently abrogate CD4+/CD25+ cells (e.g., ONTAK or ZENAPAX), thereby enhancing the host immune response to the pathogen.

Preferred prime boost regimens for HIV can be oligomeric gp140 envelope(s) of HIV consensus or wildtype encoding sequences plus HIV gag, pol and nef encoding sequences (see, for example, U.S. Provisional Application Nos. 60/503,460 and 60/604,722 and PCT/US04/30397) preferably derived from early transmitted HIV strains, that would be primed either as DNA or recombinant adenovirus expressing the envelope/gag/pol/nef and boosted with the protein, the envelope/gag/pol/nef expressed in mycobacteria, or HIV antigens expressed in recombinant adenovirus. Alternatively the immunogen can be given repetitively to induce an immune response with ONTAK or ZENAPAX or other anti-T reg cell agent given during the prime only, during the prime and boost, or during the boost only. Other inhibitors of T regulatory cell function can be given either before or during the time of vaccine priming or boosting.

For TB vaccination, ONTAK or ZENAPAX or other regulatory T cell inhibitors can be administered either before or at the time of the vaccine, with the vaccine being either BCG, modified BCG (with the listerolysin gene for example), attenuated TB, or another vector, such as MVA or rAd that expresses protective TB vaccines.

The mode of administration of the immunogen and agent used to suppress CD4+/CD25+ function and/or number can vary with the immunogen and agent, the patient (human or non-human mammal) and the effect sought, similarly, the dose administered. Optimum dosage regimens can be readily determined by one skilled in the art. Generally, administration will be subcutaneous, intramuscular, oral, intravenous or intranasal.

It will be apparent from a reading of this disclosure that, in addition to the use of the present approach to enhance immune responses to HIV and TB vaccines, this strategy can also be used to enhance the immune response to any vaccine such as (but not limited to) recombinant anthrax protective antigen administered, for example, in alum.

The invention further relates to a method of identifying an immune response enhancing agent suitable for use in the method described herein. The method comprises screening test compounds for the ability to suppress the number of CD4⁺/CD25⁺/Foxp3+ T regulatory cells, or the immunosuppressive function of said T regulatory cells. A compound that effects such suppression is a candidate immune response enhancing agent. (Suitable model systems include those described in the Examples that follow.)

The studies described in Example 3 demonstrate that transient depletion of T-regulatory cells (using a MAb specific for CD25) enhances systemic and mucosal peptide-specific T-cell responses. Immunization along with an adjuvant induces an accelerated recovery of T-regulatory cells. TLR-ligands 3, 4, 7 and 9 were also able to induce T-regulatory recovery in the thymus and spleen. These results provide new approaches to adjuvant design in order to minimize the impact on thymopoeisis and modulating T-regulatory cells.

Certain aspects of the invention are described in greater detail in the non-limiting Examples that follows.

### EXAMPLE 1

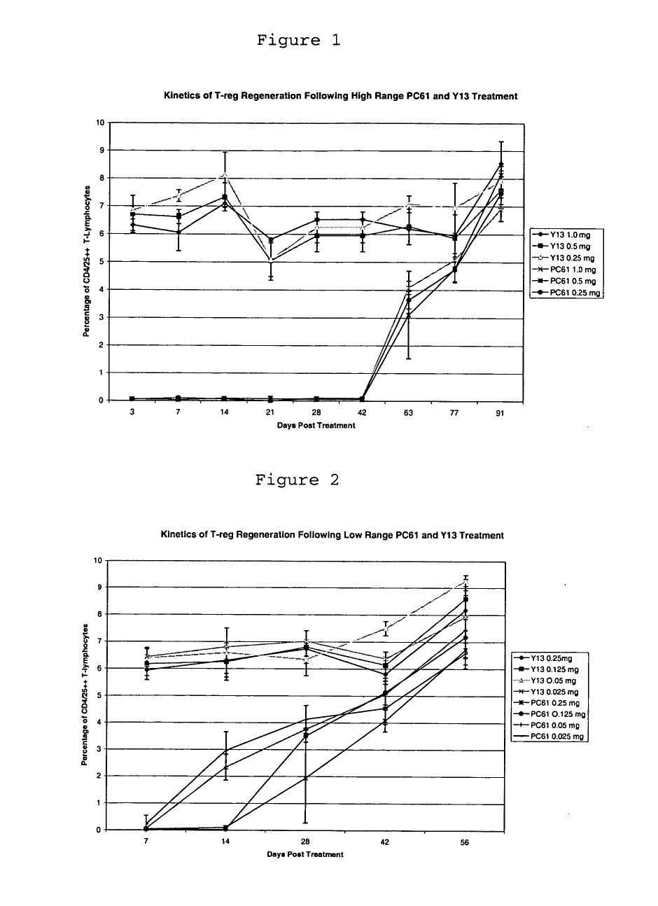

The following study was undertaken to determine the effect of the removal of T-regulatory cells on vaccine responses. PC61.5.3 is a hybridoma that produces rat anti-mouse CD25 antibodies (American Type Culture Collection, Manassas, Va.). This hybridoma was grown in Cell-Line flasks in serum free medium and the antibody was purified by ammonium sulfate cuts and finally dialyzed against PBS. A dosing experiment was undertaken to determine the amount of PC61 to be administered in order to remove CD4⁺/CD25⁺ cells from BALB/c mice. The first study used doses of PC61 and Y13 (a control rat IgG1) of 1 mg, 0.5 mg and 0.25 mg. The antibody was given intraperitoneally (IP) and three days later, spleen, thymus and whole blood were harvested from half the mice of each group. CD4⁺/CD25⁺ levels were reduced in all tissues and thus the decision was made to use whole blood to monitor the CD4⁺/CD25⁺ population in the thymus and spleen. Mice were subsequently bled at 2 week intervals. CD4⁺/CD25⁺ levels began to return to normal (control) levels after day 42 and the mice were harvested at day 91 upon the complete regeneration of CD4⁺/CD25⁺ cells. (See FIG. 1.)

A ‘low dose’ experiment was also conducted using PC61 at 0.025 mg, 0.050 mg, 0.125 mg and 0.25 mg. The experiment was undertaken to determine if lower doses of PC61 would allow CD4⁺/CD25⁺ levels to return to normal more quickly. Mice given 0.025 and 0.05 mg of PC61 began repopulation of CD4⁺/CD25⁺ cells around day 14 and reached normal (control) levels around day 42. CD4⁺/CD25⁺ levels in mice receiving 0.125 and 0.250 mg of PC61 were detectable 2 weeks later, at day 28. (See FIG. 2.) This same study was performed in C57BL/6 mice to determine if there was any strain variation. No difference in the levels of CD4⁺/CD25⁺ cells was observed as between BALB/c mice and C57BL/6 mice given ‘low dose’ levels of PC61.

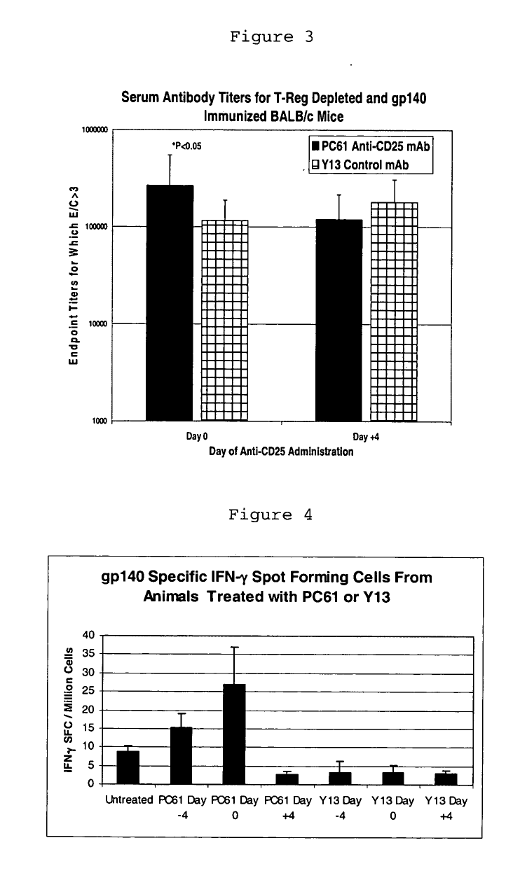

Further studies were conducted involving the administration of PC61 or Y13 in conjunction with immunization of CON6 gp140 with the 62.19 V3 sequence in it (Gao et al, J. Virol. 79:1154-63 (2005), U.S. Applications 20040086506, 20040001851, 20030219452 and/or 20030147888, U.S. Provisional Application Nos. 60/503,460 and 60/604,722). These animals were given PC61 or Y13 at a ‘High Dose’ of 0.25 mg and, in a separate experiment, a ‘Low Dose’ of 0.025 mg. PC61 or Y13 was delivered intraperitoneally 4 days prior to immunization, at the time of immunization or 4 days following immunization. The immunogen was whole protein HIV envelope gp140 delivered subcutaneously with the MPL+TDM (Ribi) adjuvant (Sigma Chemicals). Mice were immunized 5 times at three week intervals and bleed at each interval. An ELISA was performed on the sera from the animals of the ‘High Dose’ group and the dilution at which the Experimental absorbance values were 3× greater than Control absorbance values was recorded. At post-immune bleed #3 there was a significantly greater titer of serum antibodies from animals treated with PC61 at Day 0 than untreated animals or Y13 treated animals. The increased titer was not see in animals treated 4 days after immunization. (See FIG. 3.)

At the time of harvest, spleen, lung and female reproductive tract (FRT) were removed and assayed for antigen-specificity with the IFN-γ ELISpot assay (Peacock et al, J. Virol., 78:13163-13172 (2004)). There were no detectable spots in samples from lung or FRT tissue and only minimal spot formation in cells isolated from the spleens. Though there was no significant difference in the IFN-γ spot formation in any of the groups tested, there was an interesting trend showing increased IFN-γ spot formation in mice treated with PC61 on Day 0 in relation to the immunization. (See FIG. 4.)

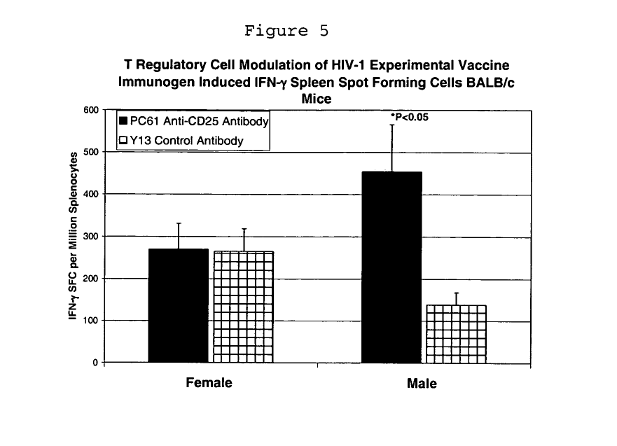

In a separate study, the effect of T-reg abrogation on the differences in antigen specific cellular responses seen between female and male BALB/c mice following intranasal administration of an HIV-1 peptide immunogen was examined (Peacock et al, J. Virol., 78:13163-13172 (2004)). In a previous study, it had been determined that male mice responded sub-optimally to nasally administered immunogen because of defective mucosal priming. Female and male mice were treated with anti-CD25 and control antibody to determine if the temporary removal of T-regs would enhance mucosal priming in males. As seen previously, males treated with control antibody (Y13) did not have an equivalent antigen-specific cellular response compared to females but males treated with PC61 anti-CD25⁺ antibody clearly overcame defective mucosal priming. (See FIG. 5.)

Summarizing, a model of CD4⁺/CD25⁺ T-reg cell depletion was developed that has made possible determination of vaccine immune responses in animals with transient depletion of T-reg cells. For this model, a rat anti-mouse CD25 IgG1 hybridoma (PC61) or an isotype matched control hybridoma (Y13) was used with multi-color flow cytometry immunophenotyping (thymus and peripheral blood) to determine the concentration needed to transiently remove the CD4+/CD25+ T-reg cell population in female BALB/c mice and to determine the kinetics of depletion. It was determined that 0.25 mg of PC61 depleted CD4+/CD25+ T-reg cells in both thymus and peripheral blood for 8 weeks. To test the hypothesis that depletion of T -reg cells would enhance immune responses to vaccination, mice were treated interperitoneally with 0.25 mg of PC61 or Y13 either 4 days prior to immunization, at the time of immunization or 4 days after immunization with HIV group M concensus envelope gp140 oligomer. Mice were immunized 5 times at three week intervals and bled at each interval. An ELISA was performed to measure serum antibody titers. Following the third immunization, significantly higher titers of anti-gp140 were observed in animals simultaneously treated with PC61 and immunogen versus Y13 control animals (265,171 GMT vs 115,914 GMT; p<0.05). Next, T-cell responses induced by a subunit vaccine given intranasally with cholera toxin—a regimen known to induce both CD4 and CD8 vaccine immune responses were examined. It has been previously determined that male BALB/c mice respond sub-optimally to this nasal subunit immunization strategy, compared with female BALB/c mice. Following treatment with PC61 at the time of immunization, male mice had antigen-specific IFN-γ ELISpot responses that were significantly higher than the Y13 control group and were comparable to female mice responses. Together these data demonstrate that transient depletion of T regulatory cells can enhance T and B cell responses to vaccination.

### EXAMPLE 2

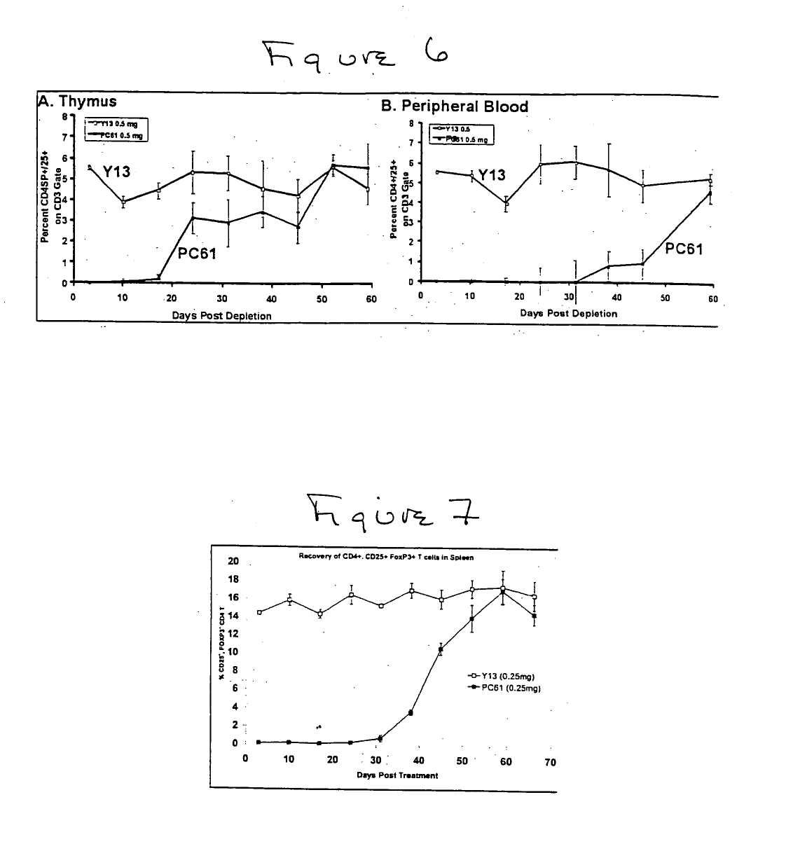

CD4⁺ T cells constitutively expressing CD25 are produced in the thymus, suppress in vivo and in vitro lymphoproliferative function and regulate the production of organ-specific autoantibodies. The hypothesis that vaccine responses in mice can be improved by transient removal of endogenous T regulatory cells (using anti-murine CD25 MAb, PC61) has been tested. It has been determined that 0.250 mg (i.p.) of PC61 depleted CD4⁺/CD25⁺ T cells in both thymus and peripheral blood for 8 weeks. Following depletion with PC61 Mab on Day 0, new CD4⁺/CD25⁺ T cells first appeared in thymus (day 24), then in spleen (day 38), followed by peripheral blood (day 45) (FIG. 6), suggesting thymic reconstitution of peripheral T regulatory phenotype cells in adult mice.

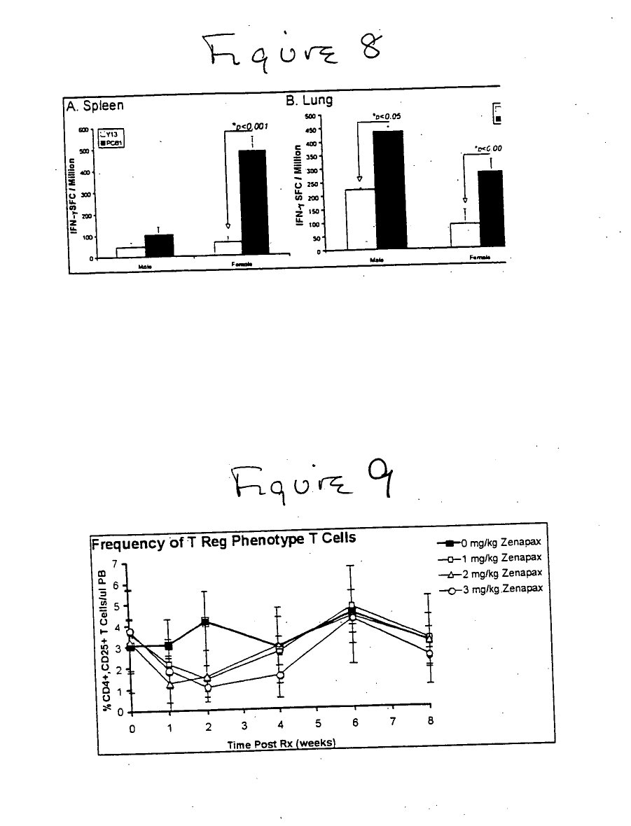

Cells from the spleen were also stained for the Foxp3 transcription factor as a surrogate marker for functional T regulatory cells. The Foxp3+ phenotype was evident by day 31 post depletion with complete reconstitution by day 52 (FIG. 7).

Next, an examination was made of Env T cell responses induced by a subunit vaccine given intranasally (i.n.) with cholera toxin (50 μg of P18 HIV epitope and 1 μg cholera toxin administered on days 0, 7, 14 and 28, the animals were sacrificed on day 35 (Peacock et al, J. Virol., 78:13163-13172 (2004))—a regimen known to induce both CD4 and CD8 immune responses. Treatment of mice with PC61 MAb at the time of intranasal immunization with Env resulted in enhanced spleen and mucosal site vaccine induction of IFN-γ spot forming cells (spleen; PC61: 611±105, Y13: 155±26, female reproductive tract; PC61: 62±27, Y13: 4±2, and lung; PC61: 1807±441, Y13: 772±614). Furthermore, it was determined that this immunization strategy induced an accelerated return of the CD4⁺/CD25⁺ phenotype T cells in peripheral blood. Following immunization, CD4⁺/CD25+ phenotype T cells were detected in peripheral blood after just 17 days whereas in unimmunized animals this recovery did not occur until 45 days.

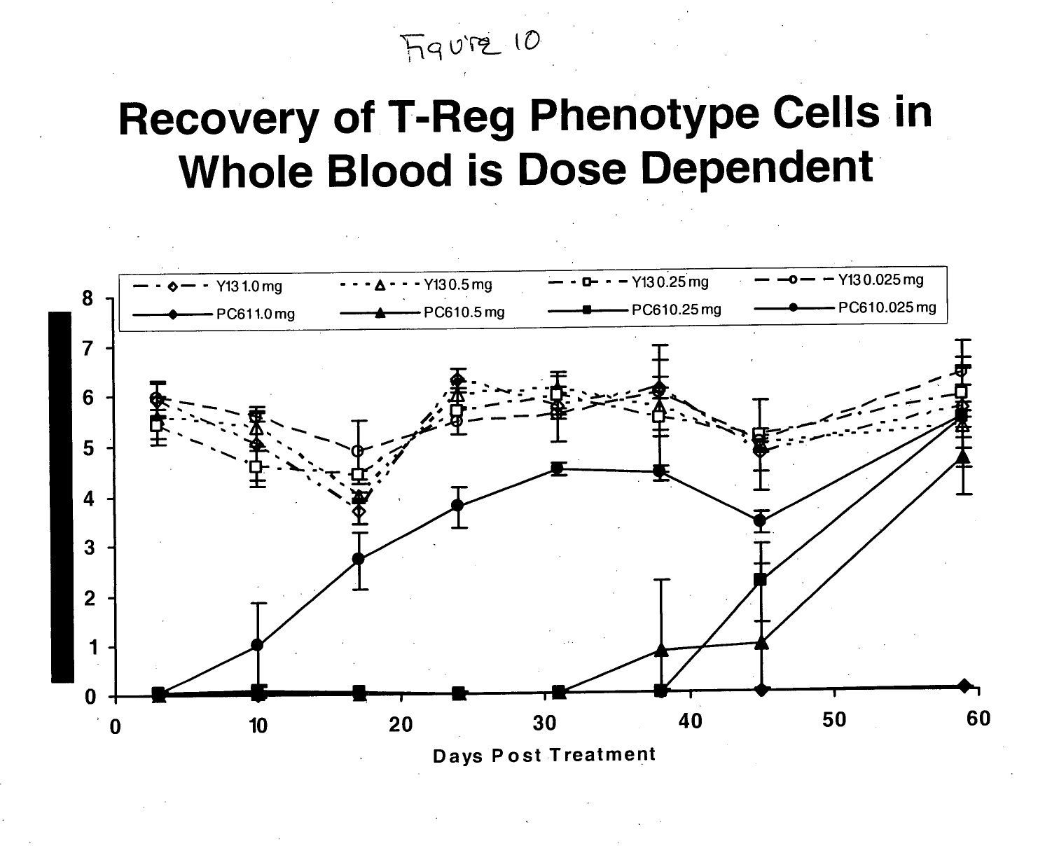

Using M. smegmatis as a surrogate, mouse model studies have been undertaken to determine the role T reg phenotype cells in host response to mycobacterium. M. smegmatis was selected because it grows rapidly, is safe for lab workers, and is vector friendly for downstream applications. The question raised was what impact does T reg cell removal (PC61 depletion vs Mab Y13 control) have on the BALB/c immune response to M. smegmatis (1×10⁷ CFU) infection. Two weeks post infection, the animals were sacrificed and the spleen, lung, serum and reproductive tract were removed for IFNγ ELIspot response to M smegmatis whole cell lysate. Significant increases in both systemic (spleen) and mucosal (lung) cellular responses were observed (FIG. 8).

A non-human primate T regulatory cell depletion model is being developed to test the efficacy of transient T regulatory cell removal in experimental select agent vaccines (rPA for anthrax). Initial dose and kinetic studies were performed with anti-hCD25Ab (Zenapax) and revealed a delayed depletion in both % and number of T regulatory phenotype cells (maximum at 2 weeks post) that lasted until 4 weeks (FIG. 9). Ontak (IL-2 diptheria toxin) will be used for dose and kinetic studies to get more rapid depletion (within 1 week).

Taken together, these data demonstrate that transient depletion of T regulatory phenotype cells can enhance both systemic and mucosal T cell responses to vaccination and that immunization induces accelerated recovery of T regulatory phenotype cells. Depletion of regulatory T cells during immunization can be a beneficial immune modulatory regimen to enhance responses to weak or suboptimal immunogens. .

### EXAMPLE 3

The studies described below demonstrate enhancement of vaccine immune responses by transient removal of T-regulatory cells and the roll of TRLs in stimulating T-regulatory cell production.

The rat hybridoma cell line that produces PC61, an IgG1 MAb specific for murine CD25, was used. The cell line is grown in serum free medium and the monoclonal antibody is purified by ammonium sulfate precipitation.

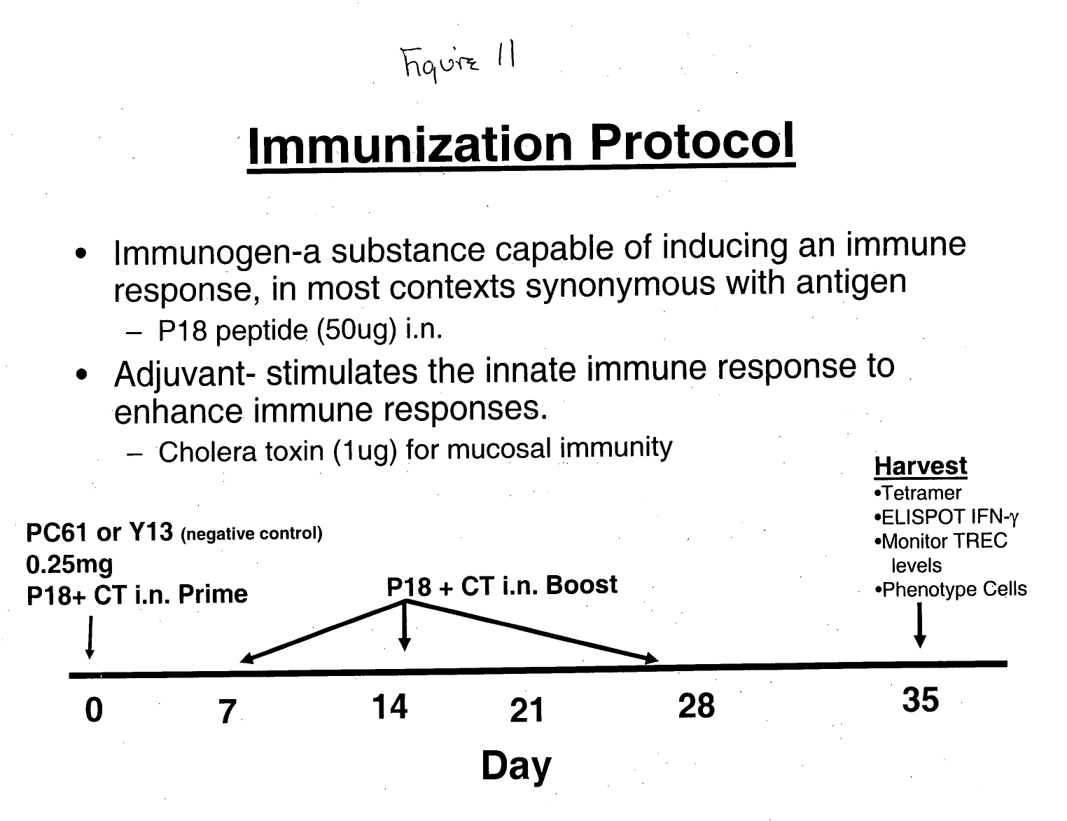

Following isolation and purification of the PC61 mAb, a determination was made as to whether the administration of the antibody interperitoneally (i.p.) would deplete CD25⁺ T cells from treated mice. PC61 and Y13, a rat IgG1 MAb used for a control, were administered on Day 0 and mice were bled 3 days following treatment. It was found that each of the concentrations of PC61 administered had effectively removed CD25⁺ T cells from the blood of treated mice while Y13 treated mice maintained normal levels of CD4⁺, CD25⁺ T lymphocytes. The duration of depletion was shown to be dose dependent with the group given the lowest concentration of PC61 (0.025 mg) recovering CD25+, CD4+ T cells one week following depletion and the group given the highest concentration (1.0 mg) of PC61 having not recovered CD25+, CD4+ T cells by the end of the study 8 weeks later. (FIG. 10.)

A determination was then made as to whether immune responses can be improved by the transient removal of endogenous regulatory T cells. As a model, an immunization strategy was used that was known for the induction of antigen-specific T-cells following intranasal (i.n.) immunization with an HIV-1 Env peptide (P18) (50 μg) with the mucosal adjuvant, cholera toxin (CT) (1 μg). The mice were primed i.n. with P18+CT on day 0 in conjunction with CD25+ T cell depletion. PC61 or the control MAb Y13 was administered i.p. at the time of the first immunization. Control groups included PC61 or Y13 control treated mice receiving P18 only and mice receiving CT only. Mice were then boosted with P18+CT on day 7, 14 and 28. On day 35 the mice were euthanized and cells isolated from the spleen and lungs were phenotyped for P18 tetramer binding. (FIG. 11.)

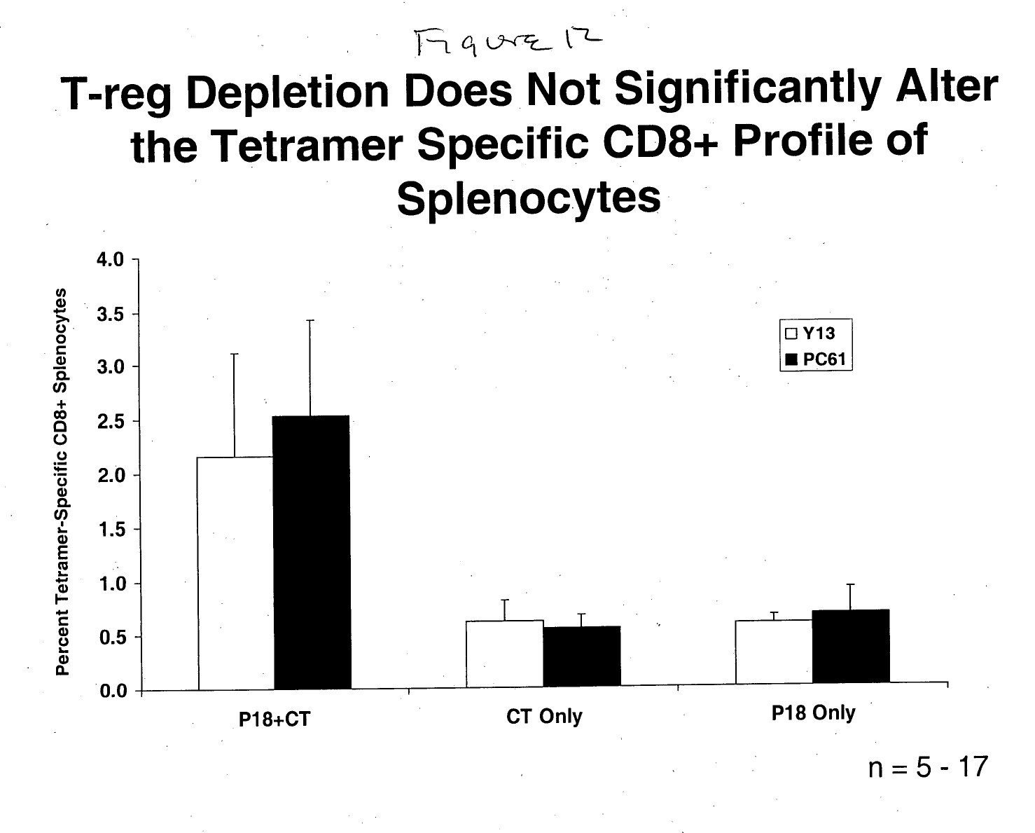

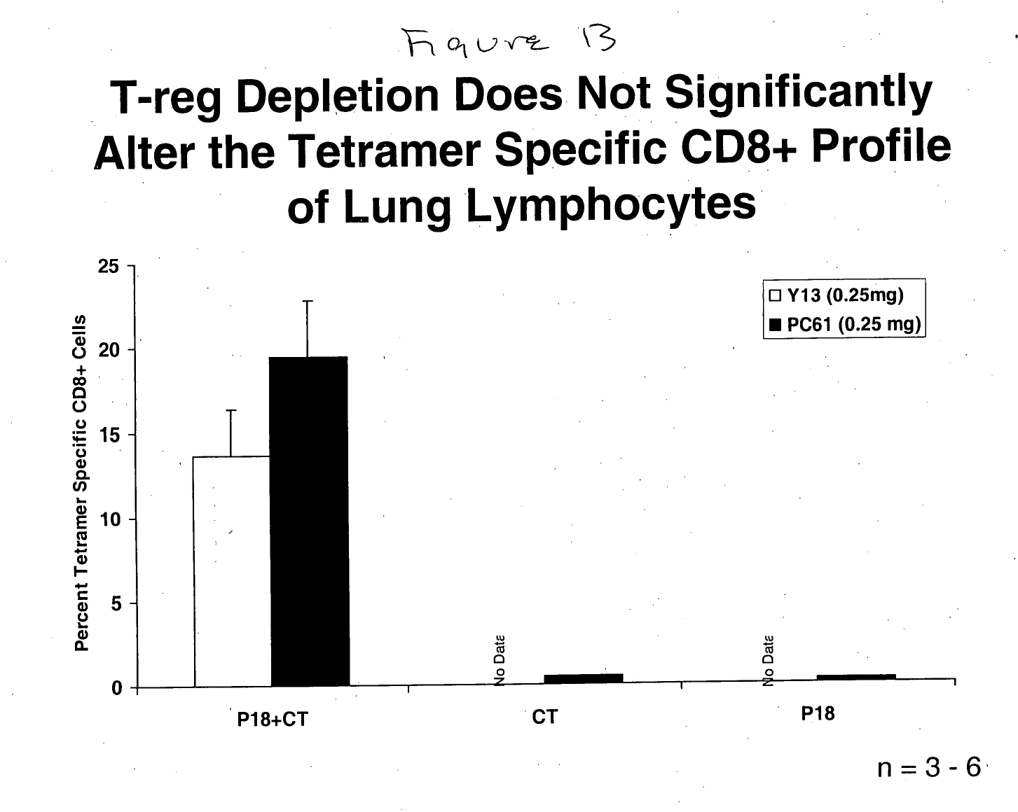

It was found that, with cells isolated from both the spleen (FIG. 12) and the lungs (FIG. 13) of immunized mice, that T-reg depletion did not significantly alter the tetramer-specific CD8+ T cell profile. However, when splenocytes were assayed in the IFN-γ ELISPOT assay, it was found that T-reg depleted mice immunized with P18+CT had significantly greater frequency of IFN-γ spot forming cells (SFC) than did Y13 control treated mice (FIG. 14). Similar results were seen in the lungs of mice immunized with P18+CT. Those T-reg depleted mice had significantly higher frequency of IFN-γ SFC than did Y13 control treated mice (FIG. 15). In both tissues, control groups of mice receiving P18 without CT or groups receiving CT without P18 had no response to the P18 re-stimulating antigen.

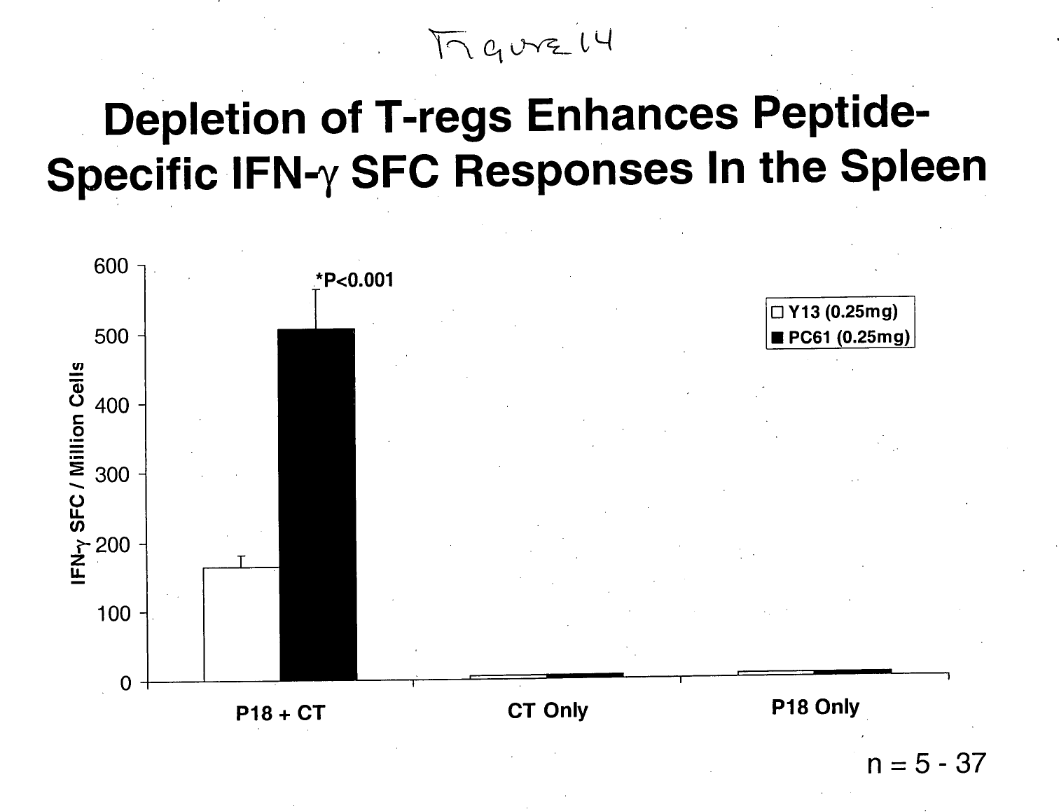

Next, an examination was made of the kinetics of T-regulatory cells in these immunized mice. Beginning 3 days after T-regulatory depletion and the first immunization of this study, mice in each of the groups were euthanized and the thymus and spleen were phenotyped with CD3, CD4, CD25, and Foxp3 to determine the rate of T-regulatory recovery in immunized mice. Again, Foxp3, a transcription factor, is considered a surrogate marker for functional T-regulatory cells in conjunction with CD25 and CD4. In the spleen, the percent of Foxp3+, CD25+, CD4+ cells in mice immunized with P18+CT had returned to normal levels by day 28 post-depletion and were significantly higher than in mice immunized with P18 only; by day 28 post-depletion Foxp3+, CD25+, CD4+ splenocytes in un-immunized mice were only beginning to recover (FIG. 16A). In addition to the percentage of Foxp3+, CD25+, CD4+ splencoytes, a calculation was also made of the absolute number of these cells in the spleen following T-regulatory depletion and immunization (FIG. 16B). The absolute number of Foxp3+, CD25+, CD4+ splenocytes follows a similar trend to the percent showing mice immunized with P18+CT displaying an accelerated rate of recovery compared to mice immunized with P18 alone or CT alone.

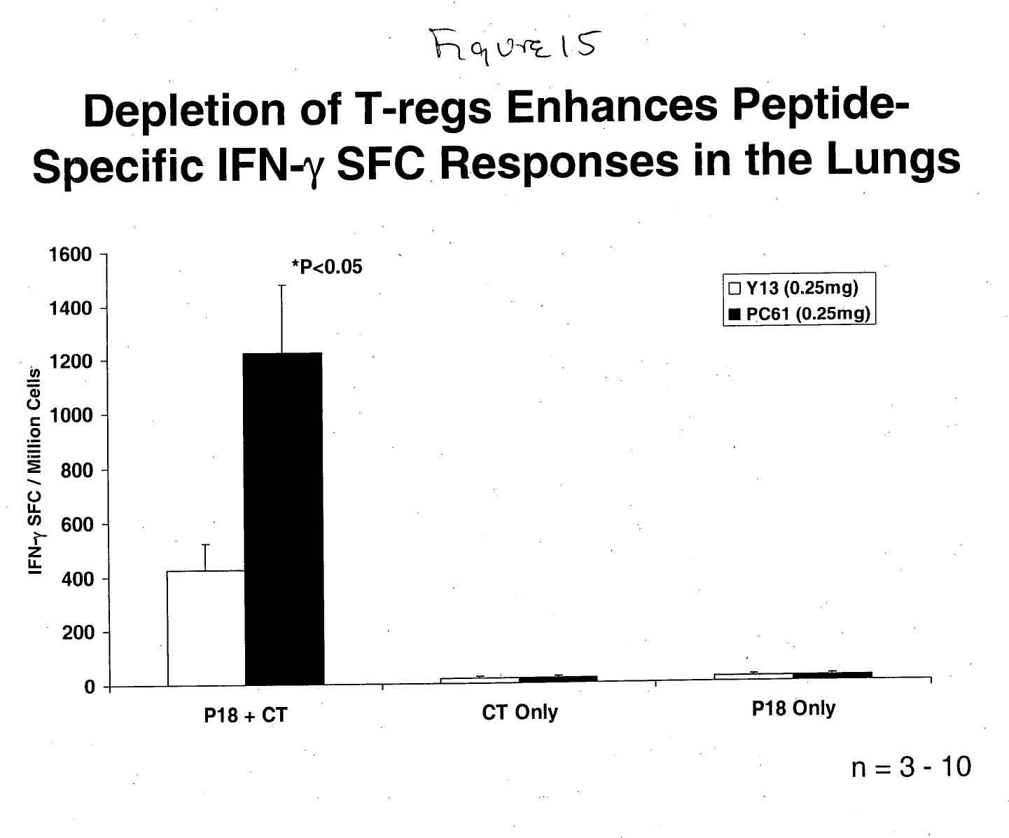

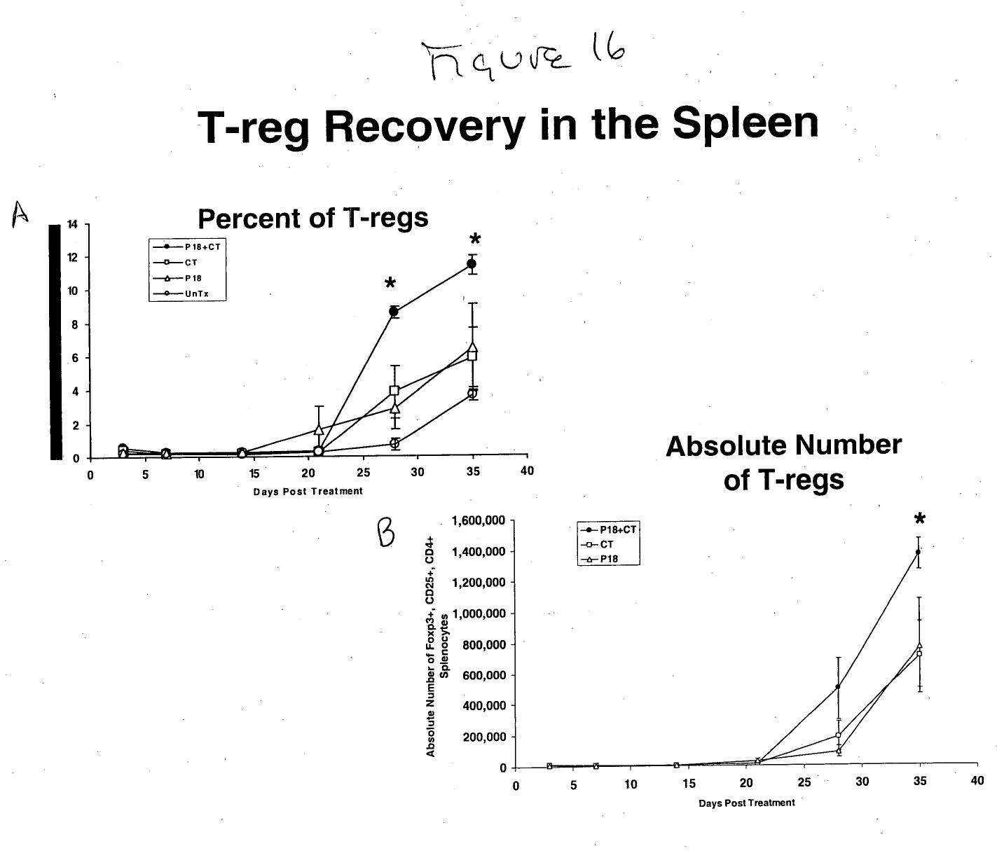

In addition to the kinetics of recovery in the spleen, a determination was also made of the kinetics of recovery in the thymus of the same mice. Again, it was found that the recovery rate of Foxp3+, CD25+, CD4 single positive thymocytes was accelerated in the T-regulatory mice immunized with P18+CT (FIG. 17A). By day 35, the percent of T-regulatory cells was significantly higher in mice immunized with P18+CT compared to mice immunized with only P18 or CT. The absolute number of T-regulatory cells in the thymus was also calculated and the kinetics of recovery appear to be similar between each group tested (FIG. 17B).

From these studies, it was found that the depletion of T-regulatory cells enhanced IFN-γ secretion by P18 peptide specific cells in the spleen and the lung while not altering the percentage of P18 peptide specific CD8+ cells as determined by tetramer phenotyping. In addition, serum samples collected 35 days post T-regulatory depletion did not have significantly elevated levels of ds-DNA, SSA, SSB, nRNP or cardiolipin auto-antibodies. It was also found that the recovery of T-regulatory cells is enhanced in mice immunized with P18 peptide in addition to the adjuvant cholera toxin.

The next question addresses was what effect other adjuvants would play in the recovery of T-regulatory cells following depletion (Medzhitov, N. Engl. J. Med. 343(5):338-344 (2000)). For this set of experiments the Ribi adjuvant was used. This is an oil-in-water mixture of bacterial and mycobacterial cell wall. Here, groups of mice were treated with PC61 (0.25 mg) or Y13 (0.25 mg) and Ribi at various dilutions (¼, 1/16 and 1/32) on day 0 (FIG. 18).

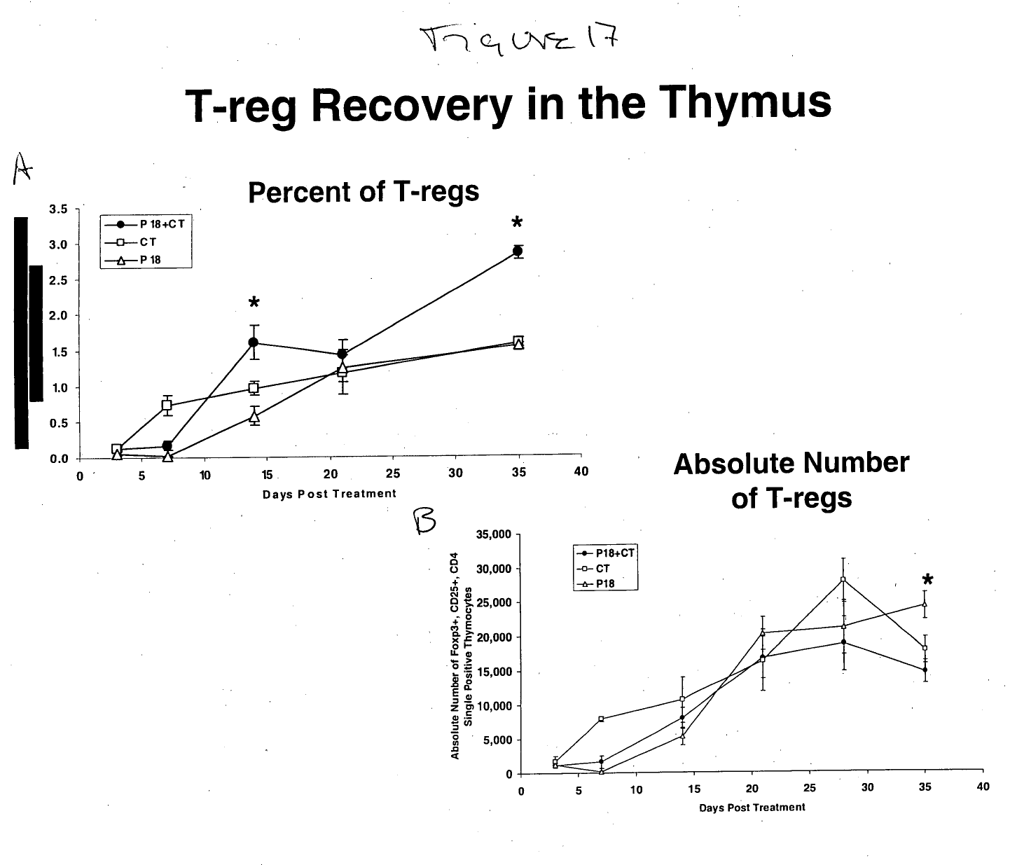

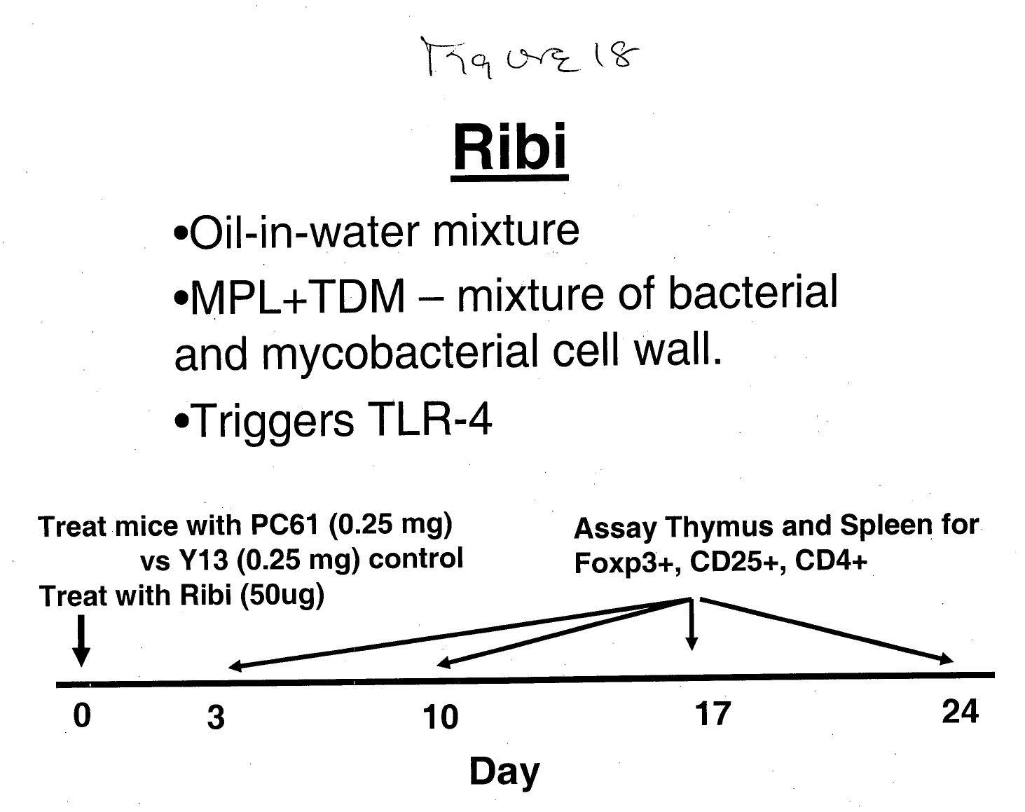

Beginning on day 3 and every 7 days following, mice were euthanized from each group and the spleen and thymus were phenotyped for Foxp3, CD25, CD4. In the thymus, the percentage of Foxp3+, CD25+, CD4 single positive T cells drops from 4.5% pre-depletion to nearly undetectable levels three days following depletion (FIG. 19A). Following this drop, there is a marked spike in the Foxp3+, CD25+, CD4 single positive thymocytes on day 10 and this spike correlates to the dilution of Ribi given. The absolute number of Foxp3+, CD25+, CD4 single positive thymocytes was also calculated and, while showing a dose-dependent rate of recovery, does not reflect the spike seen in the percent Foxp3+, CD25+, CD4 single positive T cells (FIG. 19B).

In the spleen of T-regulatory depleted mice treated with Ribi, the percent of Foxp3+, CD25+, CD4+ splenocytes dropped from a pre-depletion rate of 14% to nearly undetectable levels by day 3 (FIG. 19C). Just as in the thymus, there is a dose dependent rate of recovery of Foxp3+, CD25+, CD4+ splenocytes related to the dose of Ribi. Mice receiving undiluted Ribi had nearly 10% Foxp3+, CD25+, CD4+ splenocytes by day 10 while T-regulatory depleted groups receiving no Ribi maintained low levels of Foxp3+, CD25+, CD4+ splenocytes (<4%) until the end of the study at day 24. The absolute number of Foxp3+, CD25+, CD4+ splenocytes returned with similar kinetics as the percent (FIG. 19D).

These studies demonstrate that Ribi as an adjuvant alone can lead to an accelerated and dose dependent recovery of T-regulatory cells in the thymus and spleen. In the thymus, there is a spike in the percentage of T-regulatory cells at day 10 post treatment but not in the absolute number of T-regulatory cells. This potentially reflects an important role of Ribi on thymopoeisis. Since Ribi is a TLR-4 ligand the question next address was what effect specific TLR-ligands would have on the recovery of T-regulatory cells following depletion.

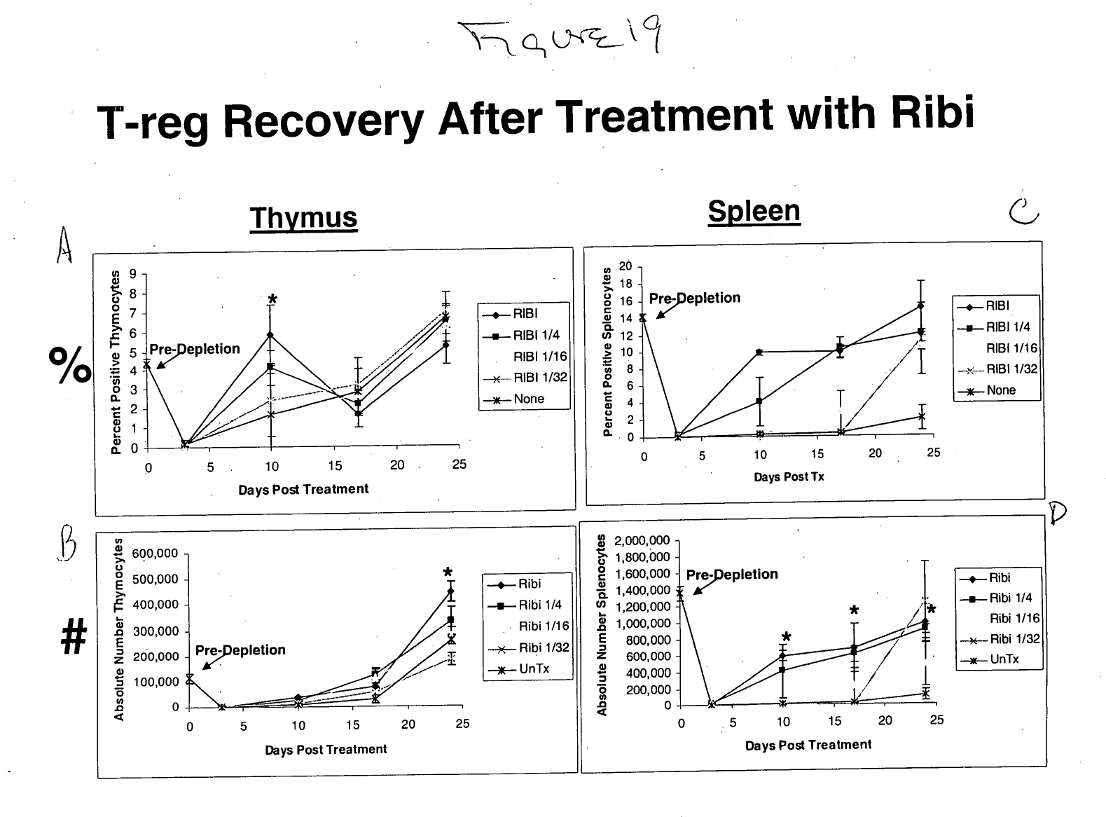

In the next set of experiments, a study was made of the effects of different TLR-ligands on the recovery of T-regulatory depleted mice. For TLR-2 ligand, LPS from P. gingivalis was used. For TLR-3 ligand, Poly (I:C) was used. Ultra pure LPS from E. coli was used as the TLR-4 ligand. For TLR-5 ligand, flagellin was used and for TLR-7, loxoribine was used. For TLR-9 ligand, oCpG was used as well as a control of oGpC. Due to the success of Ribi in previous experiments, it was again used as a positive control and untreated mice were used to determine the effects of the TLR-ligands on recovery of T-regulatory cells following depletion. As in previous studies, mice were given 0.25 mg of PC61 to deplete T-regulatory cells or the Y13 control on day 0. At the same time treatment groups received the appropriate adjuvant. Three days following treatment and every 7 days thereafter, mice were euthanized and the spleen and thymus phenotyped for the presence of T-reg cells using the Foxp3, CD25 and CD4 markers. (FIG. 20.)

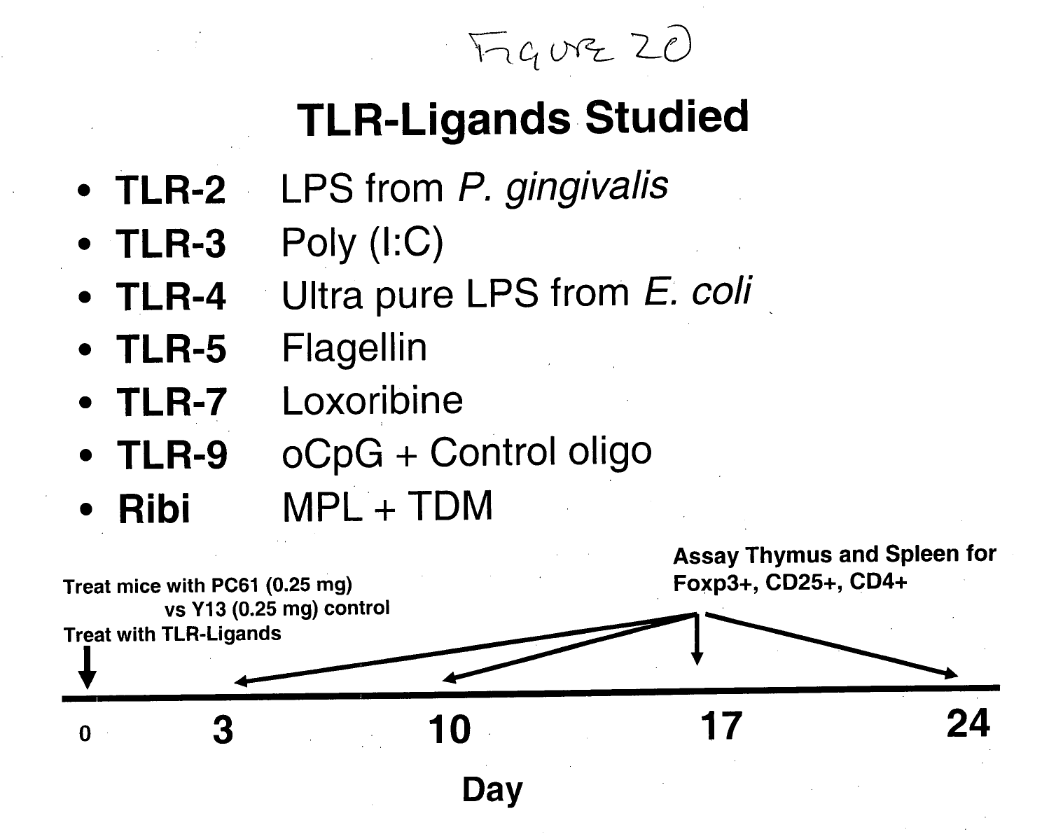

On day 3 post-treatment, the percent of Foxp3+, CD25+, CD4 single positive thymocytes were undetectable in PC61 treated mice. By day 10 there was a significant spike in the percent of Foxp3+, CD25+, CD4 single positive thymocytes in groups treated with TLR-3, 4, 7 and 9 ligands as well as those mice treated with Ribi. There was no difference at day 10 in the percent Foxp3+, CD25+, CD4 single positive thymocytes between groups treated with TLR-2, 5, 9 control and the untreated group. By day 17 the percent Foxp3+, CD25+, CD4 single positive thymocytes in mice treated with TLR-3, 4, 7, 9 ligands and Ribi had dropped to levels that were not significantly different from untreated mice. (FIG. 21.)

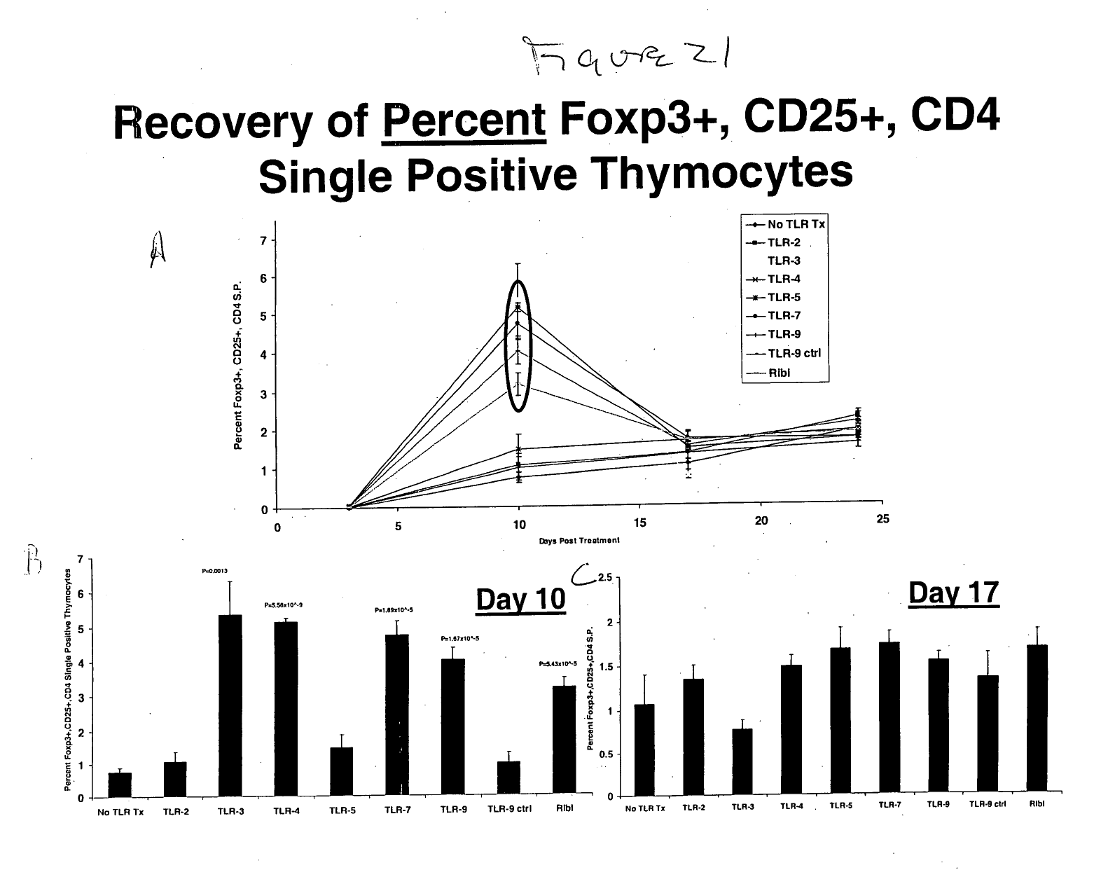

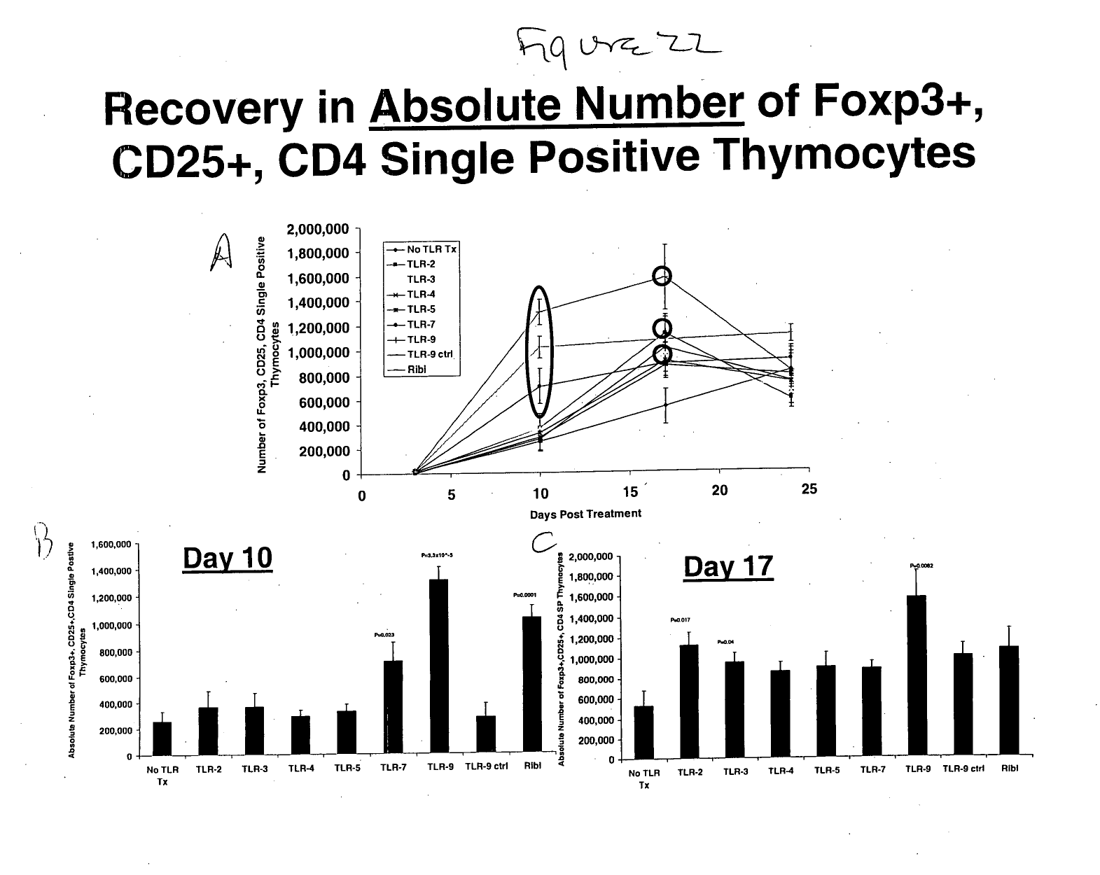

In calculating the absolute number of Foxp3+, CD25+, CD4 single positive thymocytes the kinetics were different. On day 10 the groups receiving TLR-7, 9 ligands and Ribi had significantly higher numbers of T-regulatory cells than did untreated mice. Analysis on day 17 showed that the numbers of Foxp3+, CD25+, CD4 single positive T-regulatory cells in untreated mice continued to rise steadily while the numbers in TLR-2, 3 and 9 ligand treated mice was significantly higher than in untreated mice. The spike in absolute number of Foxp3+, CD25+, CD4 single positive thymocytes appears at day 17 as opposed to the spike at day 10 in the percent Foxp3+, CD25+, CD4 single positive thymocytes suggesting that these TLR-ligands are having an effect on thymopoeisis. (FIG. 22.)

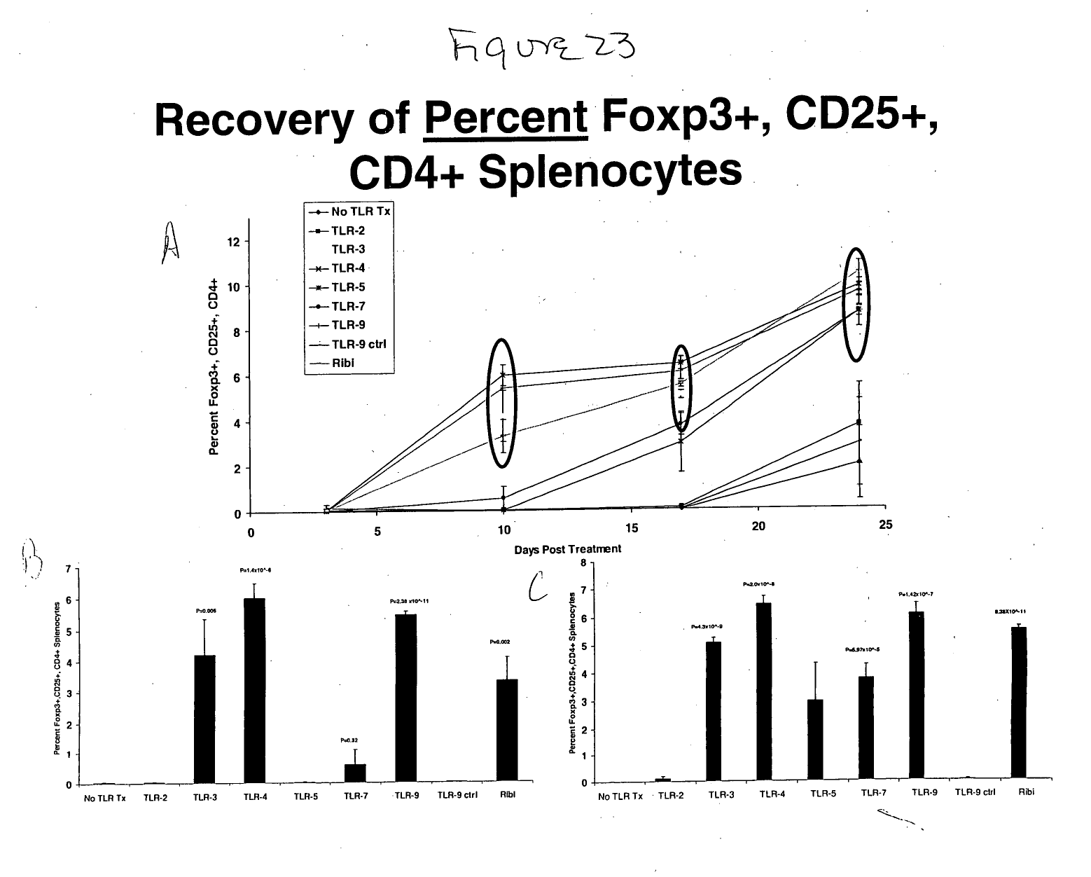

Recovery of Foxp3+, CD25+, CD4+ splenocytes was also measured in the treated groups and compared to that of T-reg depleted mice receiving no TLR-ligand. On day 3, Foxp3+, CD25+, CD4+ splenocytes were barely detectable in PC61 treated mice but by day 10 T-reg depleted mice treated with TLR- 3,4,9 ligands and Ribi had significantly higher levels of Foxp3+, CD25+, CD4+ splenocytes than untreated mice. By day 17 mice treated with TLR-ligands 3, 4, 7, 9 and Ribi had significantly increased levels of Foxp3+, CD25+, CD4+ splenocytes than untreated mice. On day 24, only groups treated with TLR-2 and TLR-9 control ligands had not returned to normal levels and were not significantly different than untreated mice. (FIG. 23.)

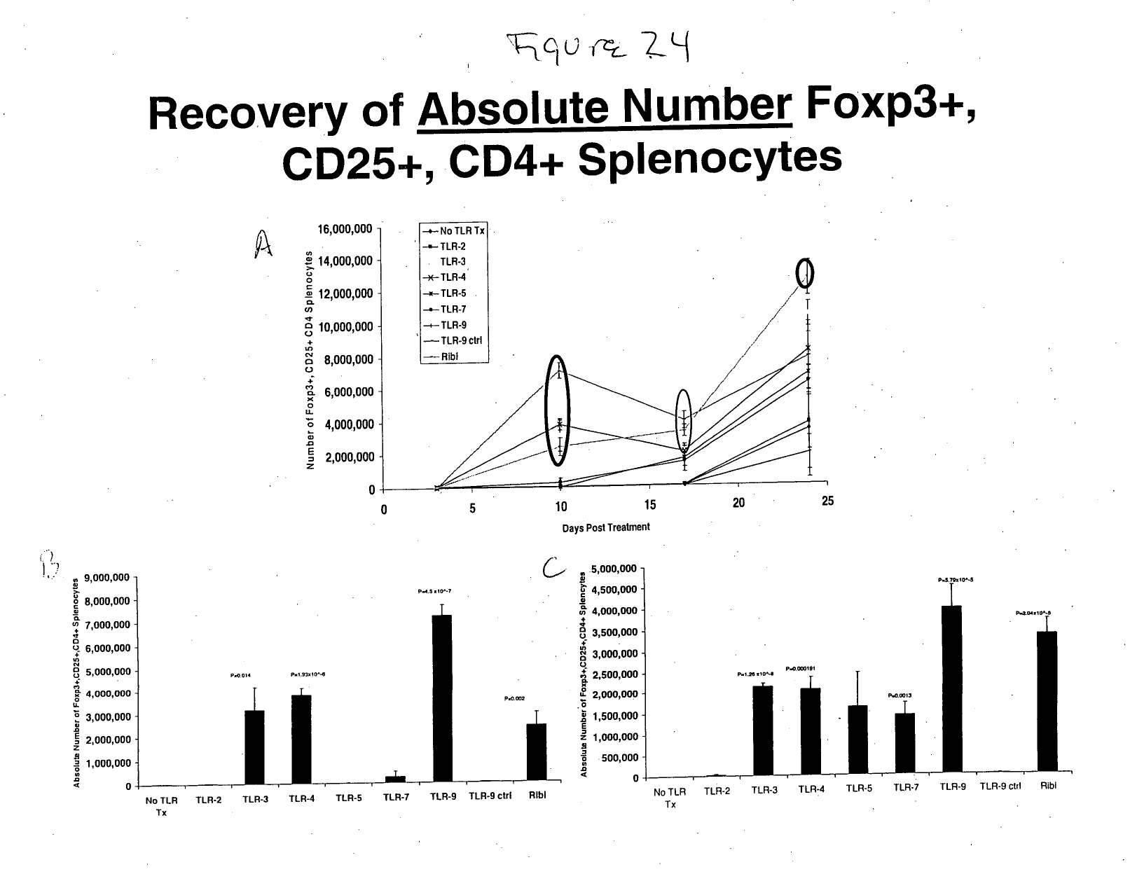

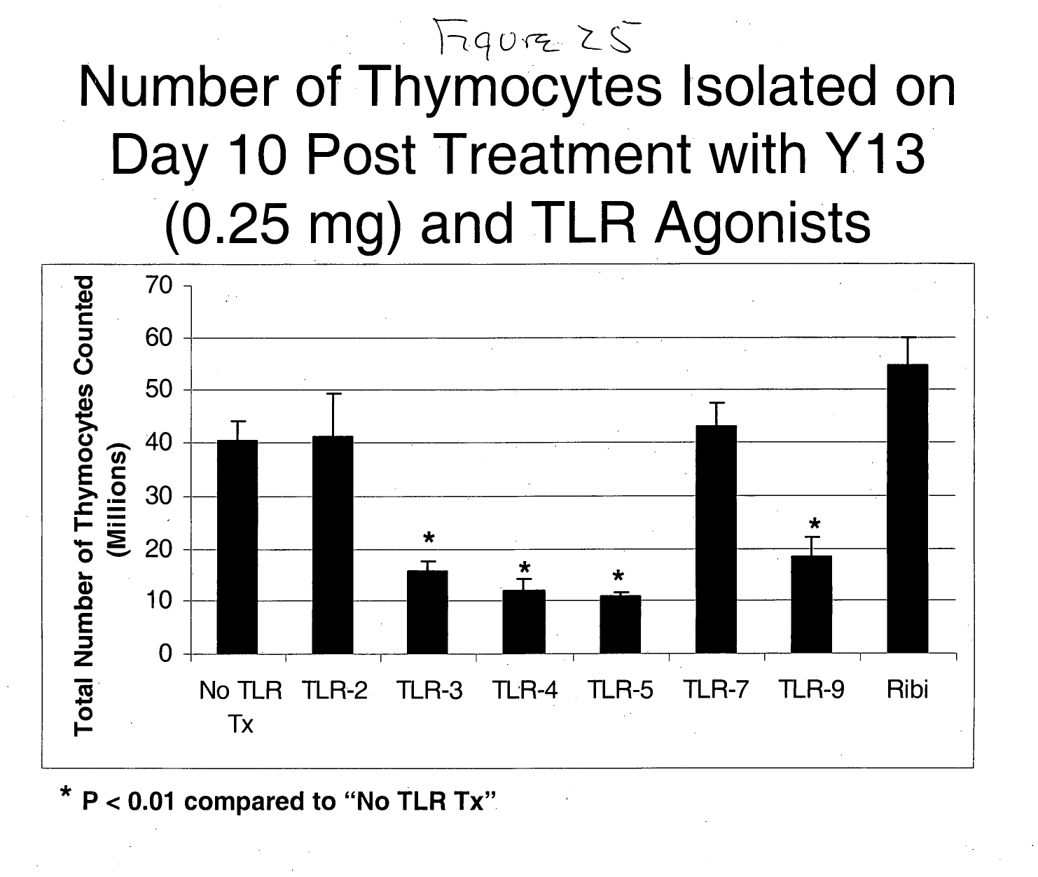

The kinetics of recovery in absolute number of Foxp3+, CD25+, CD4+ splenocytes was similar in that there were nearly undetectable levels on day 3 but by day 10 groups receiving TLR ligands 3, 4, 9 and Ribi had significantly greater levels than untreated mice. On day 17, groups treated with TLR-ligands 3, 4, 7, 9 and Ribi had significantly higher numbers of Foxp3+, CD25+, CD4+ splenocytes than the untreated group. By day 24 post depletion/treatment only one group (Ribi) had significantly higher absolute numbers of Foxp3+, CD25+, CD4+ splenocytes than did untreated mice. (FIG. 24.)

These studies show that after T-regulatory depletion there is a spike in the percentage of Foxp3+, CD25+, CD4 single positive thymocytes at day 10 after treatment with TLR-ligands 3, 4, 7, 9 and Ribi. This suggest that these TLR-ligands have an effect on thymopoeisis while TLR-2 and 5 have no effect. T-regulatory recovery is accelerated on day 10 in the thymus by TLR-ligands 7, 9 and Ribi and in the spleen by TLR-ligands 3, 4, 9 and Ribi. On day 17, T-reg recovery was greater in the thymus in groups treated with TLR-ligands 2, 3 and 9 while in the spleen T-regulatory recovery was accelerated by TLR-ligands 3, 4, 7, 9 and Ribi.

In conclusion, T-regulatory cells can be transiently depleted from the thymus and periphery with a single i.p. dose of PC61 MAb. This transient depletion enhances systemic and mucosal peptide-specific T-cell responses. In addition, it was found that immunization along with an adjuvant induces an accelerated recovery of T-regulatory cells. TLR-ligands 3, 4, 7 and 9 were also able to induce T-regulatory recovery in the thymus and spleen. Taken together, these results provide new approaches to adjuvant design in order to minimize the impact on thymopoeisis and modulating T-regulatory cells.

FIG. 25 shows number of thymocytes isolated on day 10 post treatment with Y13 control antibody and TLR agonists. Data show that TLR 3, 4, 5 and 9 suppress thymopoiesis while TLR-7 and 2 do not. moreover TLR-7 and 9 (FIG. 22B) enhance reconsititution of T regulatory cell recovery in the thymus at Day 10. Taken together these data suggest that TLR 2, 7 and 9 may be good adjuvants to both stimulate T and B cell responses with transient T regulatory cell depletion, and at the same time, enhance T regulatory return to prevent any adverse autoimmune activity.

All documents and other information sources cited above are hereby incorporated in their entirety by reference.

What is claimed is:

1. A method of enhancing an immune response in a mammal to an immunogen comprising administering to said mammal an amount of an agent that transiently suppresses the number of CD4⁺/CD25⁺/Foxp3+T regulatory cells, or the immunosuppressive function of said T regulatory cells, in said mammal sufficient to effect said enhancement.

2. The method according to claim 1 wherein said immunogen is an infectious disease immunogen.

3. The method according to claim 1 wherein the number of said T regulatory cells is suppressed.

4. The method according to claim 3 wherein said agent is an antibody.

5. The method according to claim 4 wherein said antibody binds specifically to the a subunit of a high-affinity interleukin-2 receptor expressed on the surface of activated lymphocytes.

6. The method according to claim 5 wherein said antibody is ZENAPAX.

7. The method according to claim 3 wherein said agent is diphtheria toxin conjugated to IL-2.

8. The method according to claim 7 wherein said agent is ONTAK.

9. The method according to claim 1 wherein the immunosuppressive function of said T regulatory cells is suppressed.

10. The method according to claim 9 wherein said agent inhibits Foxp3 expression or the function thereof as a transcription factor.

11. The method according to claim 10 wherein said agent is a polynucleotide.

12. The method according to claim 11 wherein said polynucleotide is an siRNA that targets Foxp3.

13. The method according to claim 10 wherein said agent is a protein or peptide.

14. The method according to claim 13 wherein said agent is a cytokine or antibody.

15. The method according to claim 9 wherein said agent blocks a cell surface molecule required for the immunosuppressive function of said T-regulatory cells.

16. The method according to claim 1 wherein said agent is coadministered with said immunogen.

17. The method according to claim 1 wherein said agent is administered prior to administration of said immunogen.

18. The method according to claim 17 wherein said agent is administered 1-7 days prior to administration of said immunogen.

19. The method according to claim 1 wherein said immunogen comprises at least one HIV envelope peptide or protein, or nucleic acid encoding said peptide or protein.

20. The method according to claim 1 wherein said immunogen is a mycobacterial or anthrax immunogen.

21. A composition comprising an immunogen, or nucleic acid encoding said immunogen, and an agent that transiently suppresses the number of CD4⁺/CD25⁺ T regulatory cells or the immunosuppressive function of said T regulatory cells.

22. A kit comprising an immunogen, or nucleic acid encoding said immunogen, and an agent that transiently suppresses the number of CD4⁺/CD25⁺ T regulatory cells or the immunosuppressive function of said T regulatory cells, disposed within at least one container means.

23. A method of identifying an immune response enhancing agent comprising screening test compounds for the ability to suppress the number of CD4⁺/CD25⁺/Foxp3+ T regulatory cells, or the immunosuppressive function of said T regulatory cells, wherein a compound that effects said suppression is a candidate immune response enhancing agent.
